(19) United States
(12) Patent Application Publication
(10) Pub. No.: US 2009/0054934 A1
(43) Pub. Date: Feb. 26, 2009

(54) EXPANDABLE FILLERS FOR BONE CEMENT

(75) Inventors: Etai Beyar, Caesarea (IL); Mordechay Beyar, Caesarea (IL); Oren Globerman, Kfar-Shemaryahu (IL); Ronen Shavit, Tel-Aviv (IL); Hila Wachsler-Avrahami, Tel-Aviv (IL)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 12/178,747

(22) Filed: Jul. 24, 2008

Related U.S. Application Data
(60) Provisional application No. 60/951,717, filed on Jul. 25, 2007.

(51) Int. Cl. A61F 5/00; A61L 24/04; A61H 1/00; A61B 19/00

(52) U.S. Cl. 606/86 R; 523/116; 118/620; 128/898; 601/1

(57) ABSTRACT

Methods, systems, and mixtures deliver a bone cement to a void in a bone, and provide an expandable filler for expanding the volume of the bone cement. The void in the bone is accessed and a bone cement material is introduced into the void in the bone. An expandable filler is introduced into the void in the bone. The introduction of the filler can take place simultaneously with the introduction of the bone cement or before or after the bone cement. The expandable filler can be expanded and the bone cement can be allowed to set.

### RELATED APPLICATIONS

The present application claims priority to U.S. Provisional Patent Application No. 60/951,717, filed on Jul. 25, 2007 and entitled Expandable Bone Filler Material, which application (along with all of the documents that it incorporates by reference), is hereby incorporated herein by reference.

The present application is related to IL patent application 166984 filed on Feb. 17, 2005 and titled “Spine Sponge”, the disclosure of which is incorporated herein by reference.

The present application is related to U.S. patent application Ser. No. 11/461,072 filed on Jul. 31, 2006 and entitled “Bone Cement and Methods of Use Thereof,” which is a Continuation-in-Part of U.S. application Ser. No. 11/360,251 filed on Feb. 22, 2006, entitled “Methods, Materials and Apparatus for Treating Bone and Other Tissue” and is also a Continuation-in Part of PCT/IL2005/000812 filed on Jul. 31, 2005. The disclosures of these applications are incorporated herein by reference.

The present application is related to PCT application PCT/IL2006/052612 filed on Jul. 31, 2006 and entitled “Bone Cement and Methods of Use thereof’ the disclosure of which is incorporated herein by reference.

The present application is related to Israel application No. 174347 filed on Mar. 16, 2006 and entitled “Bone Cement and Methods of Use thereof’ the disclosure of which is incorporated herein by reference.

### BACKGROUND

It is common to employ cement to repair bones in a variety of clinical scenarios. For example, compression fractures of the vertebrae, which are a common occurrence in older persons, cause pain and/or a shortening (or other distortion) of stature. In a procedure known as vertebroplasty cement is injected into a fractured vertebra. Vertebroplasty stabilizes the fracture and reduces pain, although it slightly restore the vertebral height and in rare cases to its original height. In vertebroplasty the cement is typically injected in a liquid phase so that resistance to injection is not too high. Liquid cement may unintentionally be injected outside of the vertebra and/or may leak out through cracks in the vertebra or into blood vessels. Such a leakage can be dangerous as it can harm adjacent nerves.

In another procedure, known as Kyphoplasty, the fracture is reduced by expanding a device, such as a balloon, inside the vertebra and then injecting the cement. Kyphoplasty reduces the risk of cement leakage by permitting a lower pressure to be used for injection of the cement, as the cement is injected into a pre-dilated void.

Published U.S. patent application 2007/0032567 to Beyar et al., the disclosure of which is incorporated herein by reference, teaches of a new type of bone filler material (commercially available as the “Confidence Spinal Cement System™” from DePuy Spine, Inc., of Raynham, Mass.) having no liquid phase and preserving a relatively stable high viscosity for several minutes immediately after mixing. These main characteristics provide a substantially safer filler material for vertebroplasty procedures with less risk of leakage, and further provide some height restoration in specific cases of Vertebral Compression Fractures (VCF).

Published U.S. patent application 2006/0122625 to Truckai et al, the disclosure of which is incorporated herein by reference, presents a new method of injecting filler material for treating VCF in which an external energy (e.g., RF) is used to change a material flow property (e.g., viscosity) during injection and/or in between two sequential injections. In a preferred embodiment, a first volume of lower viscosity filler is injected to the vertebra, then RF energy is emitted to enlarge the first volume filler viscosity, and finally a second volume of same filler is injected into to first volume, which now may serve as an expandable outer cover, which may improve both leakage durability and height restoration. Similarly, IL patent application No. 166017, the disclosure of which is fully incorporated herein by reference, describes cement introduction in two stages, when the cement of the second phase is optionally injected prior to curing of the first phase cement, in order to compact cancellous bone and/or reduce the fracture, and to strengthen the treated vertebra. Such a method can further promote some height restoration, in a similar manner to Truckai et al. application.

It is the object of this invention to provide a new filler material, device and method of use aiming at improving leakage durability and height restoration for treating VCF or other disorders, while substantially simplifying the procedural aspects.

### SUMMARY

The invention includes methods, systems, and mixtures for delivering a bone cement to a void in a bone, and providing an expandable filler for expanding the volume of the bone cement. According to a first aspect, the invention provides a method for filling a void in a bone. This method includes accessing the void in the bone and introducing bone cement into the void in the bone. An expandable filler is introduced into the void in the bone. The introduction of the filler can take place simultaneously with the introduction of the bone cement or before or after the bone cement. The expandable filler can be expanded and the bone cement can be allowed to set. In one embodiment, the bone is a vertebra.

In a further aspect of the invention, a mixture for filling a void in a bone and restoring its height is provided. The mixture includes an acrylic bone cement and an expandable filler, where the filler can be expanded to expand the bone cement to restore the height of the bone having the void.

In a further aspect of the invention, a system for filling a void in a bone and restoring its height is provided. The system includes a mixture of an acrylic bone cement and an expandable filler that can be expanded to expand the bone cement to restore the height of the bone having the void. The system further includes an energy source that can be selectively applied to expand the filler to expand the bone cement and restore the height of the bone having the void.

In embodiments of each aspect, the bone can be a vertebra.

### BRIEF DESCRIPTION OF THE DRAWINGS

The invention will be more fully understood from the following detailed description taken in conjunction with the accompanying drawings:

FIG. 1 provides a flow chart of a method according to the invention;

FIGS. 2A and 2B illustrate a directable cannula in a straight condition and the cannula tube of the directable cannula in a curved condition, respectively;

FIG. 3A is a cross-sectional view of a mesh structure introducing system useful with the invention;

FIG. 3B illustrates an apparatus similar to that of FIG. 3A in which a guiding cannula serves as the delivery device of the bone cement material;

FIGS. 4A and 4B show a collapsed formation and an expanded formation of permeable walled structure, respectively, of introducers useful with exemplary embodiments of the invention;

FIGS. 5A and 5B, illustrate a permeable element wall containing several through holes to permit flow or extrusion of bone cement material into cancellous bone and/or into a cavity formed in the vertebral body;

FIGS. 6A-6E show an exemplary set of instruments for introducing bone cement and filler according to the invention;

FIGS. 7A-7D show another exemplary set of instruments for introducing bone cement and filler according to the invention;

FIGS. 7E-7G illustrate method steps according to the invention for injecting bone cement and filler using the instruments of FIGS. 7A-7D;

FIG. 8 illustrates the application of an energy source to expand the filler according to the invention; and

FIG. 9 illustrates the use of a water swelling solute as the expandable filler.

### DETAILED DESCRIPTION

A broad aspect of the invention relates to a bone void filler material characterized by a volumetric change after a mass portion of it was introduced into body. Preferably, said volumetric change is expansion. Optionally, the overall filler expansion is accomplished by expansion of at least one component of said filler material and/or a specific region or part of it. In an exemplary embodiment of the invention, the filler material is introduced and expands within a bone cavity or inner volume; optionally said bone is cancellous, optionally it is a vertebra.

In an exemplary embodiment of the invention, the expansion is unidirectional and/or uniaxially. Optionally, the filler material is injected specifically towards the upper and/or lower vertebral end plate(s) in order to improve height restoration. Optionally, a unidirectional expandable volume is injected in the middle of the implanted cement. Optionally, said volume is substantially spherical. Optionally, said volume is formed of a material that expands when temperature is rising.

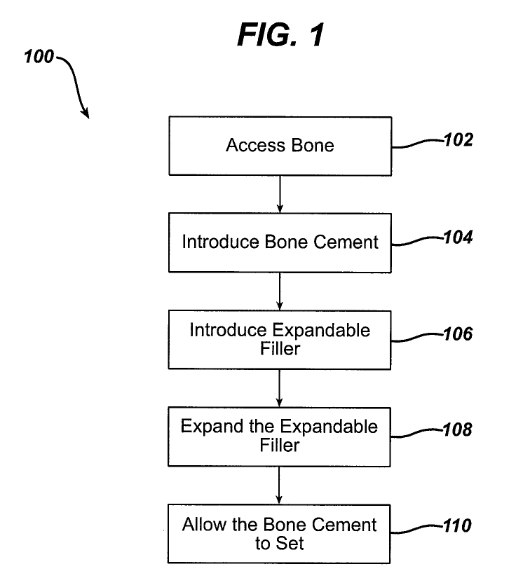

FIG. 1 provides an exemplary method 10 according to the invention. First, a surgeon gains access 12 to a bone to be treated. In preferred embodiments, the bone treated is a vertebral body. A bone cement is then introduced 14 to the bone being treated, and an expandable filler is also introduced 16 to the bone. As explained in detail below, the bone cement may include the expandable filler (or even be the expandable filler), or bone cement and expandable filler may be separate elements that are introduced in any order that is convenient into the bone. In addition, the bone cement and expandable filler may be, but do not have to be, introduced into the interior of the bone to be treated. In one embodiment, for example, the bone cement and expandable filler could be introduced between adjacent vertebral bodies whether or not they penetrated into the interior of either vertebral body. Once the expandable filler is introduced to the bone, the expandable filler is expanded 18. After expansion, the bone cement is allowed 20 to set in the expanded position.

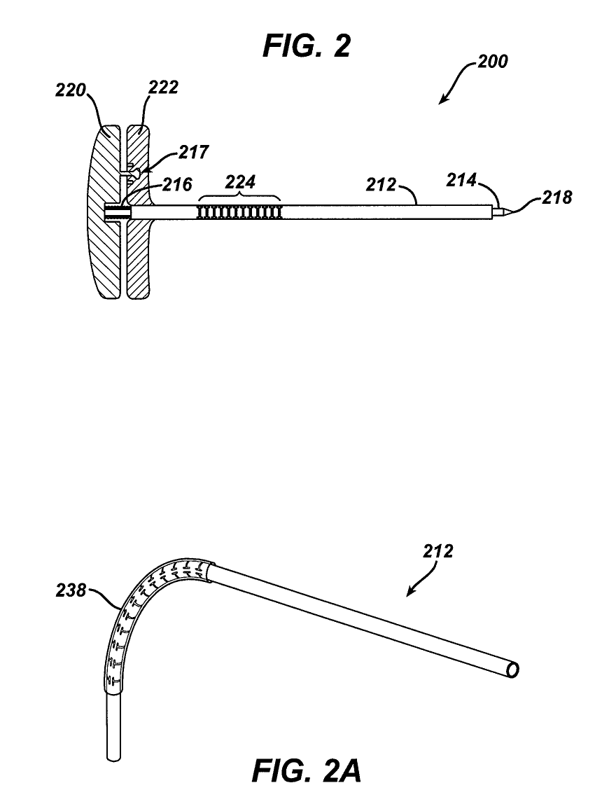

In an exemplary embodiment of the invention, a filler material is introduced into bone via a cannula having a directional opening for lateral injection, thus a directional injection may also assist in controlling the cement expansion location and/or direction. One such cannula useful for introducing bone cement and or expandable filler to a bone is illustrated in FIG. 2. FIG. 2 is a front view of an assembled Cannula/stylet apparatus 200 useful with the invention, showing a partial cross-section view of handles thereof. As illustrated, the handle orientation can match to the slit orientation (described below), so that in typical use, the forces applied by a doctor to insert the cannula will not be in the same direction as forces that are used to bend the cannula. Optionally, the handle direction is used to indicate the desired deformation direction.

Cannula 212 includes a series of slits 224 designed to impart a desired plastic deformation capability to a specific portion of the cannula. Cannula 212 optionally includes a handle 222 at its proximal end.

Stylet 214 is inserted through cannula 212 via an inner lumen of the cannula. A cutting tip 218 of stylet 214 can protrude from a distal end of cannula 212. Distal tip 218 can be adapted to puncture and penetrate the skin, soft tissue and/or cortical bone. Tip 218 may be, for example, of diamond type, drill type, bevel type or J-type, or of other tip types known in the art. Optionally, a distal tip of the cannula is formed of a radio opaque material of different opacity and/or there is a step in diameter between the cannula and the stylet, so that transition is clearer on an x-ray image.

Stylet 214 can be equipped with a proximal handle 220. In an exemplary embodiment, handles 222 and 220 engage one another via an engagement mechanism 216, for example a threaded connection. Optionally, a spring is provided to elastically couple the components. An alternative locking mechanism 217 is shown as well, in which a tongue on one handle snap-locks to a groove on the other handle. Such snap-locking may be, for example, by rotation or by axial motion.

In one embodiment of the invention, stylet 214 can be rigid. Optionally, a rigid stylet supports cannula 212 during insertion and prevents deformation of cannula 212 until such deformation is desired. In an exemplary embodiment of the invention, the stylet is removed before deformation is undertaken. A lumen of cannula 212 can be adapted to comply with a diameter of stylet 214. For example, an inner cannula lumen of 2.7 mm may be provided with a stylet of 2.6 mm.

In a further embodiment, stylet 214 can be curved. Alternatively or additionally, stylet 214 can be flexible, for example, at a portion corresponding to slit series 224.

In an exemplary embodiment, stylet 214 has a preferred orientation (e.g., is beveled) which optionally matches an angled/beveled tip of the cannula.

In an exemplary embodiment relating to the treatment of a fractured vertebral body, stylet 214 has a diameter of about 1.4-2.6 mm. It is noted that viscous material may be provided to other bone sand/or other parts of the body using the apparatus and methods described herein. The cannula optionally has an inner diameter of about 2.7 mm and an outer diameter of about 3 mm. When employed in a vertebroplasty procedure, the assembled cannula stylet 200 can be introduced into the body, so distal tip 218 penetrates skin, soft tissue and vertebra. Stylet 214 can then be disconnected from cannula 212, which remains in situ for delivery of bone cement and/or filler as described above.

As illustrated in FIG. 2A, the is a perspective view of a cannula 212 fitted with a sleeve 238 to prevent leakage of cement injected through the cannula. Sleeve 238 is deployed to cover the slits. While the sleeve is depicted on the outside of the cannula, it may optionally be provided as an inner coating. Alternatively or additionally, an external coating may be applied to cannula 212 to reduce leakage. In an exemplary embodiment, sleeve 238 adheres to cannula 212 with sufficient force to prevent or reduce leakage of bone cement being injected at pressures in the range of 100 to 300 (or 50 to 200) atmospheres. Optionally, sleeve 238 extends beyond the portion of the cannula which is slit. In an exemplary embodiment, sleeve 238 is non-compliant so that during cement injection at high pressure, the sleeve diameter remains the same. Optionally, sleeve 238 is made of a polymer with sufficient wall thickness for stability under the relevant injection pressure. Optionally, sleeve 238 is placed over cannula 212 during use (e.g., after insertion of the cannula, or prior thereto). Optionally, cannula 212 is provided with sleeve 238 in place. In an exemplary embodiment, the slit cannula provides mechanical support for the sleeve, which may be, for example, coated on or adhered to the cannula.

Further details of an exemplary directable cannula for bone cement injection are described in U.S. patent application Ser. No. 11/468,421, the disclosure of which is incorporated herein by reference.

In an exemplary embodiment of the invention, a fenestrated cannula may be used so the bone cement material is inserted to bone as separated thin hair-like protrusions that are assembled together to a bulk mass adjacent to the cannula fenestrated area. In such a case, the assembled bulk mass within bone inherently contain air pockets and/or bubbles, that will tend to expand when heated, thus promote expansion of the overall filler material mass. Bone cement material containing entrapped air pockets may also be prepared by mixing the material components with air (as in non-vacuum mixers). In addition, pockets of expandable gases other than air could be used by preparing or injecting the mixture in a gas other than air.

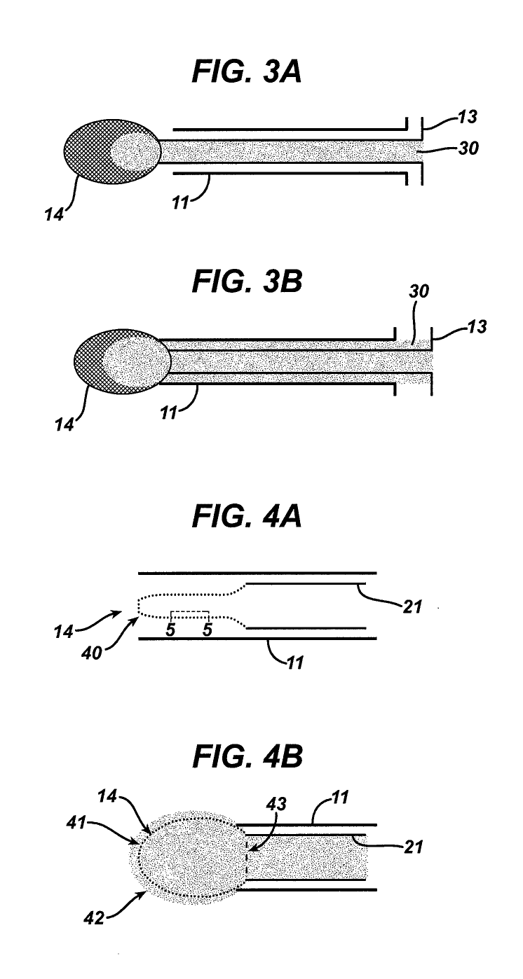

FIGS. 3A and 3B illustrate basic delivery methods and devices in accordance with exemplary embodiments. FIG. 3A is a cross-sectional view of a mesh structure introducing system, generally comprising an expandable-collapsible permeable element 14 and an extraction mechanism 13 inside the guiding cannula. As schematically illustrated, the bone cement 30 may be delivered to the permeable element directly through the extraction mechanism. The permeable element may comprise a permeable or a leak proof hollow body. FIG. 3B illustrates a similar apparatus. In this embodiment, the guiding cannula serves as the delivery device of the bone cement material. The bone cement material may flow through the location occupied by the extraction mechanism within the guiding cannula, or it may flow in the space created therebetween, depending upon the specific configuration of the extraction mechanism (including the example described with respect to FIGS. 2A-B).

FIGS. 4A and 4B show a collapsed formation and an expanded formation of permeable walled structure, respectively, in accordance with exemplary embodiments. FIG. 4A is a cross-sectional view of an expandable-collapsible permeable element 14 attached to extraction mechanism 21 inside the guiding cannula. The permeable element is shown in its first alternatively preferred collapsed formation 40. The permeable element can be positioned inside the guiding cannula either in whole or in part when collapsed until it is placed in the vertebral body prior to the injection of the bone cement material.

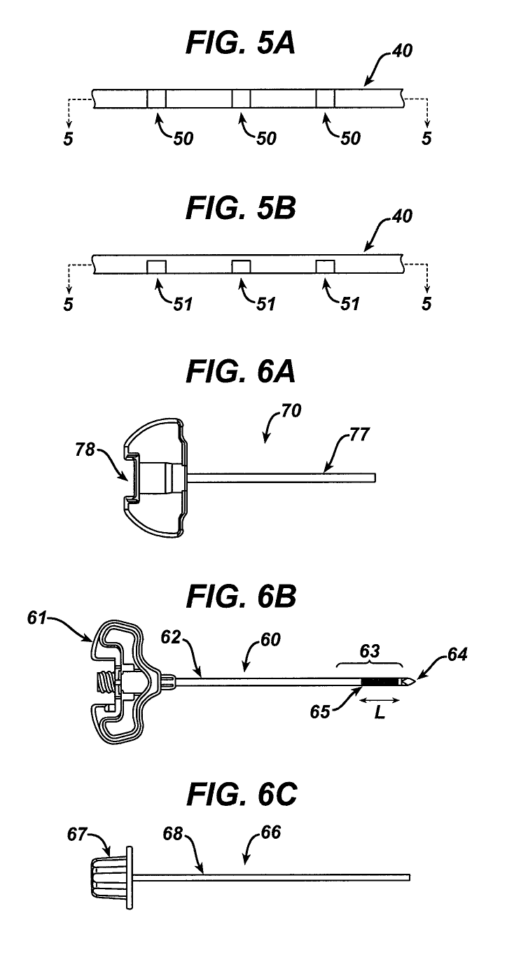

Dotted line 5-5 shows a section of the permeable element. As shown in FIGS. 5A and 5B, the permeable element wall may contain several through holes 50 or “blind” holes 51. These holes permit flow or extrusion of bone cement material into cancellous bone and/or into a cavity formed in the vertebral body. In an exemplary embodiment of the invention, the diameter of the holes may range from about 0.1 mm to about 0.5 mm. Alternatively, the flow or extrusion from the holes may occur only after the permeable element has expanded to its preferred formation configuration. Preferably, the flow or extrusion may occur only during extraction of the permeable element out of the vertebral body into the distal opening: of the cannula.

The blind hole or holes of the permeable element are preferably closed and may be capable of being burst by the bone void filler when a higher inner-pressure is achieved and after the permeable element has expanded to a preferred size or configuration. Alternatively, the hole(s) of the permeable element may be open and have certain diameter or size, which permits flowing or exudation of the bone cement material with certain properties and only after a preferable inner-pressure is met. The diameter and size of the holes may vary. Alternatively, a hole's diameter and/or shape may be changed before, during, or after expansion and/or injection of bone cement.

Preferably, the inner-pressure of the permeable element may be developed when or after the permeable element has expanded to a preferred size or configuration and is extracted from the vertebral body. The diameter of the holes may range from about 0.1 mm to about 0.5 mm. The inner-pressures may exceed 20 to 300 Atmospheres. In one embodiment of the invention, the holes may be located in specific areas of the permeable element thereby permitting a flowing of bone cement to a specific location in vertebral body and/or in a specific flowing direction.

FIG. 4B illustrates another configuration of the permeable element after it has expanded to another preferred expanded formation 41. As schematically illustrated, the bone cement material has filled the volume enclosed by the permeable element and is shown as it emerges through the holes. Preferably, the bone cement material is delivered to the permeable element through an opening port 43.

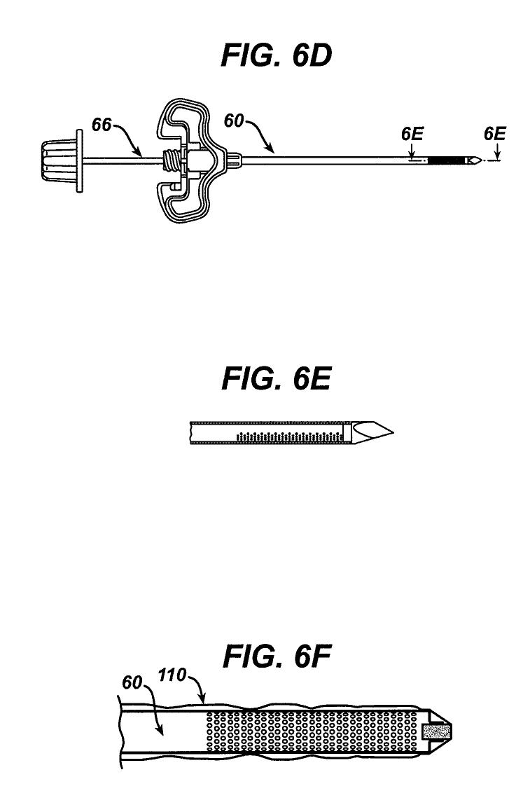

FIGS. 6A-6E show an exemplary set of instruments that can be used for VCF treatment. The set comprises a guiding cannula 70 (shown in FIG. 6A), a fenestrated cannula 60 (shown in FIG. 6B), and an inner rod/stylet 66 (shown in FIG. 6C). The cannula 60 and the inner rod 66 may be assembled (as shown in FIG. 6D) prior to insertion into the body. Generally, the inner rod 66 may be used, when a further hardening of the cannula is needed (e.g., improved bending durability) during insertion into the bone. The guiding cannula 70 generally comprises a handle 77 and a body 78 and may be made of any rigid biocompatible material (e.g. stainless steel).

The cannula 6.0 comprises a handle 61 and a body 62 having a distal end 63. The cannula 60 may be made of any rigid biocompatible material (e.g. stainless steel). Preferably, the cannula body 78 may be made long enough to reach the inner volume of a vertebra during posterior and/or anterior surgeries. A perforated area with plurality of pores 65 may be placed along at least part of the cannula distal end 63. Alternatively, there may be at least 2 pores, or at least 10 pores, or at least 50 pores, or at least 100 pores, or at least 200 pores, or at least 500 pores. In one exemplary embodiment of the invention, the area of the pores has a length L of about 1 mm, or about 10 mm, or about 20 mm, or about 40 mm or lesser, or greater, or of intermediate values. Alternatively, the area of the pores may cover a full rotation around the longitudinal axis of the cannula 60 (not shown). Alternatively, the area of the pores may cover less than a full rotation around the same longitudinal axis (as shown in FIG. 6E). In one exemplary embodiment of the invention, the diameter of each pore may be about 0.1 mm, or about 0.3 mm, or about 0.5 mm, or lesser, or greater, or of intermediate values.

Alternatively, the cannula 60 may be sealed at its distal end, so that the bone cement material may be delivered only through the pores 65. Alternatively, a shaped tip 64 may be incorporated into the cannula's distal end, thus creating a seal therewith. Alternatively, the shaped tip may be specifically designed for allowing particular functionality. In exemplary embodiments, the shaped tip may be designed as a trocar, and/or a driller, and/or a reamer, thus enhancing bone access capabilities of the present invention.

The inner rod 66 comprises a handle 67 and a rod 68. When assembled, the distal tip of the inner rod and the proximal end of the shaped tip are close to one another (not shown), and optionally in contact. Alternatively, the handles 61 and 67 may be capable of being interconnected.

In an exemplary method of treatment (not shown), the assembled set is introduced into a vertebra until a preferred portion of the cannula's distal end has penetrated to the desired location The inner rod is then withdrawn. The bone cement material may then be pressurized into the cannula towards its distal end. After injection, the cannula may be withdrawn from the body.

In an alternative embodiment shown in FIG. 6F, the fenestrated cannula 60 may be combined with a longitudinal sleeve cover 110. Alternatively, the cannula and the sleeve cover may be connected at least to one point and/or a curve and/or an area. They may be alternatively connected at least at their distal tips. Another alternative may be to crimp the tips together.

In another embodiment, the sleeve cover may be at least partially made from a mesh structure (e.g. knitted/weaved fabric) and/or from a perforated membrane. If a mesh structure is used, it may be appropriate to use fibers having good resistance to tensile strength (e.g. stainless steel, high performance synthetic fibers, etc). Other biocompatible fibers, such as plastic (e.g. PMMA) fibers, may also be used.

When the bone cement material is injected into the bone using the injection device described herein, the sleeve cover is expanded before and/or during extrusion of the bone filler material into its surroundings. Injection of the bone cement material by embodiments of the present invention promotes homogeneous interdigitation within the bone and/or around the perforated segment.

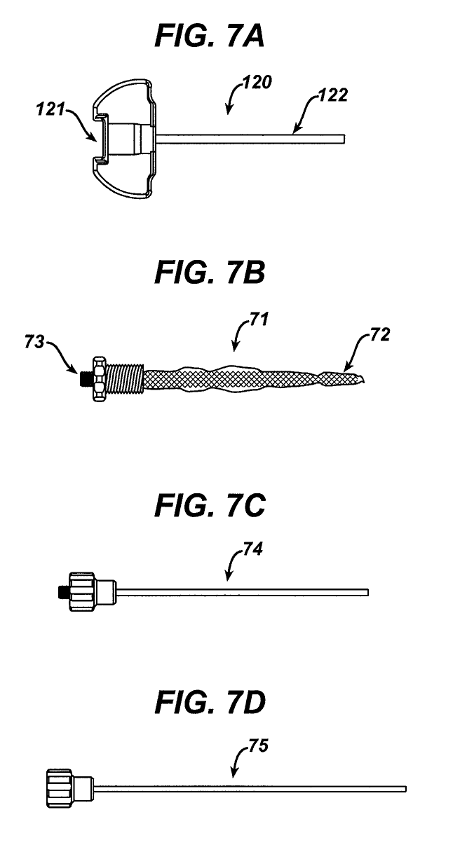

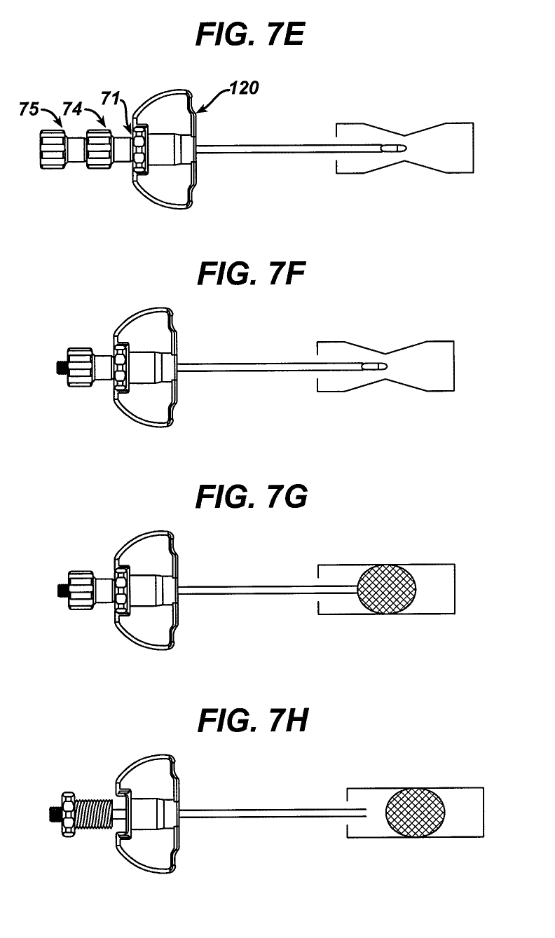

FIGS. 7A-7D show another exemplary set of instruments that can be used for VCF treatment. The set comprises a cannula 120 (shown in FIG. 7A), a longitudinal sleeve 71 (shown in FIG. 7B), an injection needle 74 (shown in FIG. 7C) and a stylet 75 (shown in FIG. 7D).

The cannula 120 comprises a handle 121 and a body 122 and may be made of any rigid biocompatible material (e.g. stainless steel). Preferably, the cannula body 122 is long enough to reach the inner volume of a vertebra during posterior and/or anterior surgeries. In one exemplary embodiment of the invention, the cannula body 122 is longer than about 50 mm, or longer than about 100 mm, or longer than about 150 mm. Alternatively, the cannula body may be approximately 120 mm long. In one exemplary embodiment, the cannula body has an outer diameter of about 2 mm, or about 4 mm, or about 6 mm, or lesser, or greater, or of intermediate values. Alternatively, the outer diameter of the cannula body may be approximately 4.2 mm. Alternative, the inner diameter of the cannula body may be smaller from its outer diameter by about 0.1 mm, or about 0.5 mm, or about 2 mm. Alternatively, the inner diameter of the cannula body may be about 3.6 mm.

The sleeve 71 comprises a handle 73 and a body 72. In one exemplary embodiment, the sleeve body 72 may be at least partially made from a mesh structure (e.g. knitted/weaved fabric) and/or a perforated membrane. If a mesh structure is used, it is most appropriate to use fibers having a good resistance to tensile strength (e.g. stainless steel, high performance synthetic fibers, etc). Other biocompatible fibers, such as PMMA fibers, may also be used. Alternatively, the sleeve handle may be coupled to the guiding cannula handle 121.

Alternatively, the injection needle 74 may be longer than the cannula body 122. The stylet 75 may be alternatively longer than the needle 74. Preferably, when the stylet is introduced into the sleeve, it may be capable of stretching the sleeve 71 to a predetermined length along its longitudinal axis, and optionally through injection needle 74 to the inner lumen. Optionally, said delivery system further includes an advance mechanism, capable of advancing and/or withdrawing the sleeve within the guiding cannula along its lumen.

In one embodiment, the advance mechanism may include at least two interconnected elements that permit relative uni-axial motion between them (e.g., a bolt-nut mechanism). For example, one element (e.g., a nut) may be fixed to the proximal end of the guiding cannula, and a second element (e.g., a mating bolt) may be connected to the proximal side of the sleeve. In that manner, the sleeve may travel distally or proximally, according to the set relative motion between the at least two interconnected elements.

The following steps are part of a complete exemplary procedure. At least a portion of these steps may be an exemplary embodiment of method of the invention. An example of steps for filling bone voids is:

(1) Positioning a patient for penetrating the guiding cannula 120 into a vertebra;

(2) Inserting a stylet 75 within an injection needle 74 which is within a sleeve 71 in a cannula 120 until at least part of the distal end of the sleeve is emerging out of the distal opening of the cannula 120 (as shown in FIG. 7E);

(3) Withdrawing the stylet out of the body (shown in FIG. 7F);

(4) Optionally, partly withdrawing the injection needle to a preferred position, so that a preferred length of the distal end of the sleeve loosely settles within the vertebra (not shown);

(5) Introducing bone cement material under pressure and in the presence of air or another gas, either mixed with the bone cement or present in the region in which the bone cement is injected, into the injection needle so that the material is urged towards the distal end of the sleeve. The bone cement material should be viscous enough and/or the pressure applied should be high enough and/or the pressure impact should be sufficient so that the distal end of the sleeve may expand to a predetermined preferred dimension and/or size and/or configuration (as shown in FIG. 7G). Preferably, the maximal diameter of the expanded part of the sleeve should be larger than the inner diameter of the guiding cannula. Alternatively, the maximal diameter may be greater than about 5 mm, or greater than about 10 mm, or greater than about 20 mm. The maximal diameter may alternatively be about 15 mm. Preferably, the force applied by the expanded part-of the sleeve to its surroundings is high enough to move the opposing endplates of the vertebra apart. Alternatively, at least a small quantity of the bone cement material may extrude or flow through the meshed walls into the surroundings.

(6) Withdrawing the injection needle out of the body. Optionally, a preferred minimal pressure may be sustained within guiding the cannula and/or the sleeve. Alternatively, this step may be accomplished after the filler material has cured to a preferred higher average viscosity than it was during the injection step, although preferably, it has not yet totally solidified.

(7) Withdrawing the sleeve out of the body while extracting at least part of the remaining bone filler material through its meshed walls (as shown in FIG. 7H). Preferably, when the expanded part of the sleeve has maximal diameter within the vertebra and when it is larger than the inner diameter of the guiding cannula, at least part of the filler material that is entrapped therein is extruded when the sleeve 71 is extracted through the cannula.

(8) Withdrawing the guiding cannula out of the body.

Again, as noted above, when done in the presence of air or another expandable gas (or when the bone cement has been mixed with such a gas), gas pockets will remain in the bone cement that can be expanded in subsequent steps.

In an exemplary embodiment of the invention, a specific quantity and/or mass of the filler material may expand about 5%, optionally about 10%, optionally about 20%, optionally about 50%, optionally about 100% from its original volume.

The bone cement material used may be of any bone cement type or any biocompatible filler material. Optionally, said bone cement material is acrylic bone cement, produced by mixing at least two components, one of which contains at least Polymethylmethacrylate powder and the other contains at least a liquid Methylmethacrylate monomer.

Generally, acrylic cements go through independent polymerization process from the mixing start, so the mixed material becomes more viscous over time until it sets to full hardness, that is similar to bone hardness. Different compositions may lead to different polymerization behaviors/curves, however all acrylic cements have two main phases after mixing: the “working phase”, when the cement is liquid and/or doughy so it can be manipulated into bone and/or interdigitate within a cancellous bone, and the “setting phase”, when the cement polymerization accelerates until full hardness. In an exemplary embodiment of the invention, the filler material (the air or other gas in the examples above) expands after it is introduced into bone and before and/or during its setting phase.

Optionally, the filler material expands when energy is emitted from an energy source external to body. Alternatively, the filler material self expands independently to any external energy source radiation. Optionally, an external energy source is used and the filler material contains at least one component that is sensitive to said energy and expands and/or initiate overall filler expansion when it absorbs a minimal radiation amount. Said energy may be one of the following: radiofrequency (RF), heat, light (coherent or broadband), including laser and IR, ultrasound, microwave, electrical and/or magnetic. Optionally, the energy source is located outside the patient body; alternatively, it can be inserted with or as part of the tool(s) inserted into the body during the procedure (e.g., an injection needle/cannula).

During the setting phase, the heat emitted from the exothermic curing process of cement may raise the cement temperature to 70-140° C. In an exemplary embodiment, the filler material self expands when it absorbs heat from its surroundings within body. Optionally, self-expansion occurs when the curing process of the acrylic filler material reaches a minimal higher temperature, for example at the beginning of the setting phase. Preferably, said temperature is higher than 37° C., optionally higher than 50° C., optionally higher than 70° C., optionally higher than 120° C. In a further exemplary embodiment, the filler expansion absorbs at least part of the heat emitted during the curing process so that the temperature remains relatively small, preferably not substantially higher than 37° C.

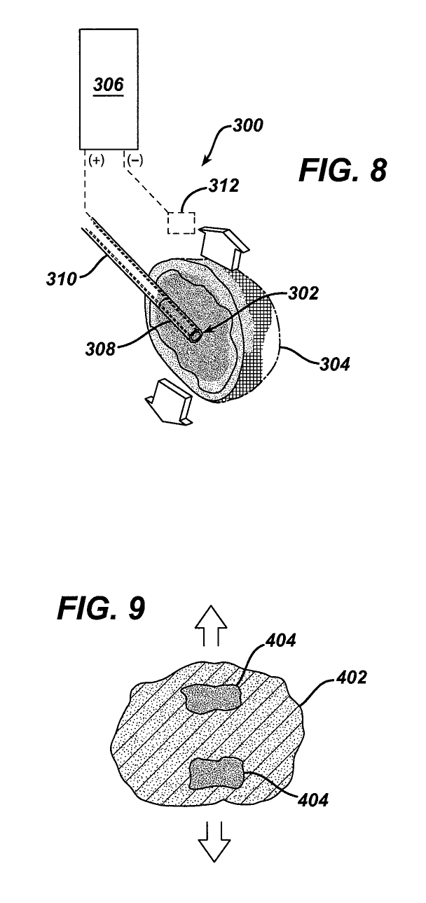

As illustrated in FIG. 8, an RF activation tool 300 can be inserted into the injected bone cement 302 in order to provide energy to expand the filler (in this case, air and/or another expandable gas mixed with the cement) and ultimately the bone cement material. The RF energy provided to heat the filler can be provided during or after delivery of the bone cement 302 into the bone 304. RF activation tool 300 includes an RF electrical source 306 to cause RF current delivery from at least one electrode emitter 308 to cause ohmic heating of the filler. In this embodiment, the distal end of a hollow introducer needle 310 carries the electrode or emitter 308. In one embodiment, the body of needle 310 is conductive while proximal portions are coated with an insulator so that only the distal portion acts as an electrode. A grounding pad 312 is also provided. As indicated in the Figure, heating continues until the filler, and concomitant with that the bone cement 302, expands. Further details of the application of RF energy to bone cement materials can be found in US published patent no. 2006/0122625 to Truckai et al., which is hereby incorporated by reference for that purpose.

The scope of the method further includes applying RF energy in multiple intervals or contemporaneous with a continuous flow of bone cement material. The scope of the method also includes applying RF in conjunction with imaging means to prevent unwanted flows or expansion of the fill material. The scope of the invention also includes applying RF energy to polymerize and accelerate hardening of the entire fill volume after the desired amount of bone cement material has been injected into a bone.

In another exemplary embodiment of the invention, the filler material expands when it absorbs fluids from its surroundings within body, from an aqueous cement mixture, or from water added specifically for the purpose of expanding the filler material. Exemplary water absorbent materials include a low molecular weight water-soluble linear polyacrylamide polymer (nominal weight average molecular weight=1500) as a preferred material. Alternatively, the filler can comprise a “cocktail” of solutes, that is, with two or more different solutes, each of which contributes different attributes to the device. For instance, one can use a solute blend of a low molecular weight solute for quick expansion of the cement and a high molecular weight solute to provide long-term pressure and stability to the cement once it is expanded and is setting. Further details of water swellable solutes useful with the invention can be found in U.S. Pat. No. 6,692,528 to Ward et al, the disclosure of which is fully incorporated herein by reference.

FIG. 9 illustrates a viscous bone cement 402 into which two pockets of water swellable solute 404 have been injected as a filler, for example, using the directable cannula described above, after injection of the bone cement. Absorption of water, for example, from an aqueous cement mixture of from water injected for this purpose, causes the cement to expand as indicated.

In an exemplary embodiment of the invention, at least one of the cement components or additives produces or discharges gas that can promote overall cement expansion. Optionally, said gas discharging occurs on a predetermined temperature or time-from-mixing.

In yet another exemplary embodiment of the invention, the filler material expands after a specific period of time since mixing start. Optionally, expansion occurs more than 3 minutes, optionally more than 5 minutes, optionally more than 10 minutes, optionally more than 15 minutes after mixing start of the filler material components. Said period of time may then set the working time boundaries of the procedure with said filler material. Alternatively, the expansion occurs few days after implantation. Optionally, the injected cement is a non-hardening cement.

In an exemplary embodiment of the invention, a bone cement material is introduced into a bone (e.g., a vertebral body) with an expandable, optionally initially compressed, sponge material. The sponge may be formerly soaked and/or saturated with said bone cement material, or alternatively may be introduced separately into the bone before, after, or simultaneously with the bone cement. Optionally, the sponge is introduced via a small diameter cannula (for example having 1-5 mm diameter, optionally about 3 mm diameter), while in compressed mode, and then expands to a larger size. Optionally, said sponge is introduced into the bone without any filler material. Optionally, a filler material is injected only for fixating the sponge to its surroundings. Such exemplary embodiment were formerly introduced in IL patent application 166984 to Etai Beyar, the disclosure of which is incorporated herein by reference. In a further exemplary embodiment, a sponge is formed of a porous shape-memory material such as the titanium alloys known commercially as Nitinol. Such materials can be designed to remember a particular shape at body temperature (or a higher temperature brought on by curing cement or external energy supplied specifically for the purpose of such heating), so that the sponge can be the expandable filler that is supplied with the bone cement.

The present invention further includes a method of treating bone (e.g., vertebra) fractures using expandable void filler material as described above. In an exemplary embodiment of the invention, after inserting (e.g., injecting) a preferred amount of said filler material into bone, the material may then be expanded, either selectively by the operator or by self-expansion, either by activating an energy source external to body or by its absorbing of energy (e.g., heat) or fluids from the surroundings within body, the expanded filler may then contribute to height restoration and/or stability of the implant within bone.

A person of ordinary skill in the art will appreciate further features and advantages of the invention based on the above-described embodiments. Accordingly, the invention is not to be limited by what has been particularly shown and described, except as indicated by the appended claims or those ultimately provided. All publications and references cited herein are expressly incorporated herein by reference in their entirety.

1. A method for filling a void in a bone comprising:
(a) accessing the void in the bone;
(b) introducing bone cement into the void in the bone;
(c) introducing an expandable filler into the void in the bone;
(d) expanding the expandable filler; and
(e) allowing the bone cement to set.

2. The method of claim 1, wherein the bone is a vertebra.

3. The method of claim 1, wherein expanding the expandable filler includes selectively applying energy from outside a body of a patient during treatment.

4. The method of claim 3, wherein the energy applied consists of radiofrequency, heat, light, ultrasound, microwave, electrical, magnetic, or combinations thereof.

5. The method of claim 1, wherein expanding the expandable filler includes exposing the filler to heat emitted during curing of the bone to cause the expansion of the filler.

6. The method of claim 1, wherein expanding the expandable filler includes exposing the filler to fluid to cause the expansion of the filler.

7. The method of claim 1, wherein expanding the expandable filler restores the height of a damaged vertebra.

8. The method of claim 1, wherein the bone cement and the filler are introduced at the same time.

9. The method of claim 8, wherein the expandable filler is a gas.

10. The method of claim 9, wherein the gas is mixed with the bone cement before injection.

11. The method of claim 9, wherein the gas is mixed with the bone cement during injection of the bone cement into the void.

12. The method of claim 8, wherein the filler is an expandable sponge.

13. The method of claim 1, wherein the bone cement has no liquid phase and remains in a stable high viscosity state after mixing and before becoming substantially set.

14. A mixture for filling a void in a bone and restoring its height comprising:
an acrylic bone cement; and
an expandable filler;
wherein the filler can be expanded to expand the bone cement to restore the height of the bone having the void.

15. The mixture of claim 14, wherein the expandable filler is a gas.

16. The mixture of claim 14, wherein the expandable filler is a water swellable solute.

17. The mixture of claim 14, wherein the expandable filler is an expanding sponge.

18. The mixture of claim 14, wherein the acrylic bone cement has no liquid phase and remains in a stable high viscosity state after mixing and before becoming substantially set.

19. A system for filling a void in a bone and restoring its height comprising:
the mixture of claim 16; and
an energy source, the energy source being selectively applicable to expand the filler to expand the bone cement and restore the height of the bone having the void.

20. The system of claim 19, wherein the energy source is a radio frequency energy source.
